(19) United States
(12) Patent Application Publication
(10) Pub. No.: US 2024/0022807 A1
(43) Pub. Date: Jan. 18, 2024

(54) ADVANCED VIDEO CONFERENCING DEVICE

(71) Applicant: Anshuman Garg, Pune (IN)

(72) Inventor: Anshuman Garg, Pune (IN)

(21) Appl. No.: 18/352,859

(22) Filed: Jul. 14, 2023

(30) Foreign Application Priority Data
Jul. 14, 2022 (IN) 202221034142

(51) Int. Cl. H04N 23/611; H04N 23/69; H04N 23/695; G08B 13/196; H04L 12/18; H04L 65/1089; H04L 65/1069; G10L 25/78; G10L 25/66; G10L 17/22

(52) U.S. Cl. CPC H04N 23/611; H04N 23/69; H04N 23/695; G08B 13/19613; H04L 12/1822; H04L 65/1089; H04L 65/1069; G10L 25/78; G10L 25/66; G10L 17/22; G16H 10/60

(57) ABSTRACT

The proposed invention provides a system (100) to facilitate humans especially the elderly/disable care. An advanced video conferencing system having processing capabilities of a smart phone with camera (8) and wired/wireless interface for establishing communicative coupling with display unit to familiar elderly/disabled people with latest communication technology. The system (100) includes a mobile computing device (1), a smart box (2) with data capturing unit (5) and an output unit (4) for video conferencing. The mobile computing device (1) receives user's input and send it to data capturing unit (5). The microprocessor of data capturing unit (5) processes the users input to control the camera (8) and/or microphone (9) to start or stop capturing voice and/or images (15, 16) of all users and send it to output unit (4). The output unit (4) receives voice and/or images (15,16) of users taking part in video conferencing and display it.

### FIELD OF THE INVENTION

The present invention generally relates to an advanced video conferencing (VC) system. More particularly, the present disclosure relates to a video conferencing system synchronized with mobile computing device to facilitate elders/disabled people care.

### BACKGROUND OF THE INVENTION

Technology has come a long way over the last 20 years or so. For example, the internet has opened up so many doors for people of all ages, making it easier for everyone to shop, work, and learn. Most young people find it easy to use modern technology because they have grown up using it. Older/disable people, on the other hand, are generally less inclined to use modern technology. Some older people might not see the ways technology could benefit them, especially if they've never used the internet or a smartphone before. While they may be purchasing laptops, smart phones and tablets and all of the possibilities they intend, many older adults say they still don't feel confident about using them.

According to the study, researchers found that many times “frustration” with new technology made older/disabled adults unsure of their ability to use it, leaving them unmotivated to even try. Frustration appeared to be a significant barrier, which led to a lack of self-confidence and motivation to pursue using the technology. Technology classes designed specifically for older people are a great start. It's important that older people are able to pick up new technology at their own pace with whatever support they may need, rather than just assuming that everybody knows how to use it all already.

As per the National Institute for ageing USA: A very high % of seniors are socially isolated, lonely, with high risks for a variety of physical and mental conditions: high blood pressure, heart disease, obesity, a weakened immune system, anxiety, depression, cognitive decline, Alzheimer's disease, and even early death.

While many caregivers understand the seriousness of the situation they are unable to do much about it due to not living with the elder or not having enough time to spend due to their work etc. Even senior care living facilities are challenged when it comes to their staff spending quality time with the seniors.

Since the pandemic began, people have become a lot more reliant on technology to communicate with our loved ones. The Covid-19 pandemic has led to an inevitable surge in the use of digital technologies due to the social distancing norms and nationwide lockdowns. The elder/disabled people took up digital communication to dismiss the loneliness and depression. The conventional device enables a user to easily control a mobile unit through a remote-control device during a Miracast connection between the mobile terminal and a display device. The other device for video conferencing includes a meeting equipment of integrated AI camera, connects between source equipment and display device. But the conventional devices have some drawbacks. The conventional device does not provide a revolving base which auto focus the subject in the frame when the subject moves around.

Hence, there rise a requirement for a complete solution for seniors with multiple forms of live engagements, mental physical exercises monitoring/observation and entertainment etc. so that they can be productively engaged for several hours a day.

### OBJECT OF THE INVENTION

Object of the present invention is to provide a system to enable humans especially the elderly to facilitate their care and engage them in multiple activities much more easily than existing devices/system. The proposed system is having processing capabilities of a smart phone, having camera and wired/wireless interface for establishing communicative coupling with display unit to facilitate the well-being of elder/disable people.

### SUMMARY OF THE INVENTION

Technology has advanced significantly in recent years, providing numerous benefits for people of all ages. However, older and disabled individuals may be hesitant to embrace modern technology due to lack of familiarity and confidence. Frustration with new technology often discourages them from trying to learn and use it.

Besides, existing devices for communication and video conferencing have limitations, such as the lack of a revolving base for auto focus. Thus, there is a need for a comprehensive solution that includes various forms of engagement, monitoring, and entertainment for seniors, enabling them to be actively involved for extended periods each day.

In an aspect, the recent disclosure provides a system designed specifically for senior care. According to the present disclosure it consists of a mobile computing device connected to a data capturing unit, which includes a camera, microphone, and second microprocessor. The system captures and transmits voice and images of users, with the camera and microphone controlled by the second microprocessor. The data capturing unit is movable and can rotate in three-dimensional space. The camera zooms based on user positions, and the microphone adjusts to individual voices. The system detects security issues, allows user control via a remote device, and can connect to smart devices for home automation. It also has features to identify distress or pain-related keywords and sounds, triggering alerts or reaching out to emergency or healthcare services.

According to one embodiment of the advanced communication system for senior care comprises a mobile computing device having an input module for receiving a user input related to starting a video conferencing session, and a first microprocessor adapted to receive the user input; a data capturing unit coupled to the mobile computing device and remotely placed with respect to the mobile computing device, and a server through a communication network, the data capturing unit includes a camera, a microphone, and a second microprocessor, wherein the second microprocessor is adapted to receive the user input from the first microprocessor, the second microprocessor is adapted to process the user input to control the camera and/or microphone to start or stop capturing one or more first user's voice and/or images, the second microprocessor is adapted to be connected to the server to send one or more first user's voice and/or images, and to receive one or more second user's voice and/or images; and an output unit which is coupled with the second processor is adapted to receive and render one or more first user's voice and/or images, one or more second user's voice and/or images or combination thereof.

According to another embodiment of the advanced communication system for senior care, the data capturing unit is placed inside a smart box, and the smart box is coupled rotational unit adapted to rotate the smart box in three-dimensional space wherein the rotational unit is connected to one of the surface of the smart box, and adapted to rotate the smart box in a plane. This embodiment provides an efficient implementation of focusing the camera. The revolving base which auto focus the subject in the frame when the subject moves around.

According to another embodiment the rotational unit is connected to a second microprocessor which controls the rotational unit to move the smart box. The second microprocessor is communicably coupled to one or more positioning sensors, the position sensors are adapted to generate a position data of the one or more first user. The second microprocessor is adapted to receive and process the position data and adapted to control the rotational unit to move the smart box.

According to another embodiment the one or more position sensors are infrared sensors, image sensors, ultrasonic sensors, resistance-based sensors, capacitance-based sensors, or optical sensors, or combination thereof.

According to another embodiment the camera is adapted to capture one or more images of the one or more first user. The second microprocessor is adapted to process the images and is adapted to determine if a size of body representation of the one or more first user are lesser than a first threshold and to control the camera to zoom in the lens of the camera, and to determine if the size of body representation of the one or more first user are greater than a threshold and to control the camera to zoom out the lens of the camera.

According to another embodiment the second microprocessor is adapted to determine identification of more than one first users in the environment where the smart box is placed and who are located at different locations in the room, the second microprocessor is adapted to control the camera to zoom in or zoom out individually for each of the first users based on processing of the images captured by the camera for each of the first user.

According to another embodiment the microphone is communicably coupled to the second microprocessor, and adapted to capture voices of each of the first users, the second microprocessor is adapted to recognize voice of each first user based on a voice recognition model, and adapted to control the movement means to move the smart box to focus the camera onto the first user whose voice is recognized at a particular instance.

According to another embodiment one or more security sensors including a heat sensor, a smoke sensor, or a motion sensor, or combination thereof, the security sensors are coupled to the second microprocessor, and the second microprocessor processes one or more security data to compare the security data with a predefined security data to determine a security issue.

According to another embodiment a remote user device being used the second user, or the mobile computing device, or combination thereof comprises a controlling trigger adapted to be communicably coupled to the data capturing unit, the movement means, the second microprocessor, a third microprocessor or combination thereof for controlling one or more functionality of the data capturing unit, the movement means, the second microprocessor, or combination thereof.

According to another embodiment the data capturing unit is coupled to a data processing unit adapted to process the first user's voice and/or images to de-noise the first user's voice, to tune contrast and/or brightness of the first user's image, or to tune resolution of the user's images, or combination thereof before sending it to the second microprocessor.

According to another embodiment wherein a remote user device being used the second user, or the mobile computing device, or combination thereof comprises a controlling trigger adapted to be communicably coupled to the data processing unit for controlling functionality of the data processing unit.

According to another embodiment wherein the input unit is an audio capturing device adapted to capture audio, and the mobile computing device comprises a speech detection unit adapted to receive and process the audio to determine identification of the first user and/or to identify keywords spoken by the first user in the audio, and to process the keywords to generate the user input.

According to another embodiment wherein the speech detection unit is adapted to identify the keyword as a distress keyword or to process the captured sound to identify distress related sound, the first microprocessor is adapted to record the captured audio and to send to a user device being used by the second user or to an emergency service provider device being used by an emergency service provider.

According to another embodiment wherein the speech detection unit is adapted to further categorize the distress keyword to pain related keyword or to categorize distress related sound to pain related sound, the first microprocessor is adapted to receive and process the pain related keyword or the pain related sound and to reach out to a healthcare service provider, or to further categorize the distress keyword to fear related keyword or to categorize distress related sound to fear related sound, the first microprocessor is adapted to receive and process the fear related keyword or the fear related sound and to reach out to a law enforcement service provider, or combination thereof.

According to another embodiment wherein the first microprocessor is coupled to a memory unit storing a historical health record of the first user and/or a current health record of the first user, to process the historical health record of the first user and/or the current health record along with the pain related keyword or the pain related sound and to reach out to a healthcare service provider.

According to another embodiment the first microprocessor is adapted to connect to one or more image capturing device capturing images of environment inside or outside a room where the mobile computing device is placed and/or one or more motion sensor devices capturing motion data inside or outside a room where the mobile computing device is placed, to process the images and/or the motion data along with the fear related keyword or the fear related sound to detect intrusion and further to reach out to a law enforcement service provider.

According to another embodiment wherein the mobile computing device is communicably coupled to one or more smart devices, and the speech detection unit is adapted to identify keywords related to controlling the smart devices, and to process the keywords to generate a user controlling input, and the first microprocessor is adapted to receive the user controlling input and communicate the same to a controller of the device, and the controller is adapted to process the user controlling input to control the smart device.

### BRIEF DESCRIPTION OF DRAWINGS

The novel features and characteristics of the disclosure are set forth in the description. The disclosure itself, however, as well as a preferred mode of use, further objectives, and advantages thereof, will best be understood by reference to the following description of an illustrative embodiment when read in conjunction with the accompanying drawings. One or more embodiments are now described, by way of example only, with reference to the accompanying drawings wherein like reference numerals represent like elements and in which:

FIG. 1 gives advanced video conferencing (VC) system in accordance with an embodiment of the present invention.

FIG. 2 illustrates system architecture diagram regarding video conferencing session performed in accordance with an embodiment of the present invention.

FIG. 3(a)-3(c) illustrate system architecture diagrams regarding camera autofocus in accordance with an embodiment of the present invention.

FIG. 4 illustrates system architecture diagram regarding remote controlling by an user in accordance with an embodiment of the present invention.

FIG. 5 illustrates system architecture diagram regarding keywords identification and generating user input and passing information to a remote user in accordance with an embodiment of the present invention.

FIG. 6 illustrates system architecture diagram regarding control by other smart devices in accordance with an embodiment of the present invention.

### DETAILED DESCRIPTION OF DRAWINGS

For promoting an understanding of the principles of the invention, reference will now be made to the embodiment illustrated in the figures and specific language will be used to describe them. It will nevertheless be understood that no limitation of the scope of the invention is thereby intended. Such alterations and further modifications in the illustrated system, and such further applications of the principles of the invention as would normally occur to those skilled in the art are to be construed as being within the scope of the present invention.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more sub-systems or elements or structures or components preceded by “comprises . . . a” does not, without more constraints, preclude the existence of other, sub-systems, elements, structures, components, additional sub-systems, additional elements, additional structures, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this invention belongs. The system, methods, and examples provided herein are only illustrative and not intended to be limiting.

Embodiments of the present invention will be described below in detail with reference to the accompanying figures.

The claimed invention is an advanced communication system designed for senior care. The system includes a mobile computing device with an input module, a data capturing unit, remotely connected to the device and a server through a communication network. The data capturing unit consists of a camera, a microphone, and a second microprocessor. The second microprocessor processes user input from the computing device to control the camera and microphone for capturing and transmitting voice and images of users. An output unit receives and renders the voice and images of the users. The data capturing unit is housed inside a movable smart box, which can be rotated in three-dimensional space using a rotational unit. The movement of the smart box is controlled by the second microprocessor based on position data from positioning sensors. The camera can zoom in or out based on the size of the user's body representation and can individually zoom for multiple users located in different positions. The microphone captures user voices, and the second microprocessor recognizes each user's voice and adjusts the camera's focus accordingly. The system can also include security sensors that are processed by the second microprocessor to detect security issues. A controlling trigger on a remote user device can control the functionality of the data capturing unit, movement means, and the second microprocessor. A data processing unit can be coupled to the data capturing unit to process and enhance the user's voice and images. The input module can be an audio capturing device, and a speech detection unit in the computing device can identify keywords spoken by the user to generate user input. Distress keywords or sounds can trigger recording and alerting emergency service providers. The system can also detect pain or fear-related keywords or sounds and reach out to healthcare or law enforcement service providers accordingly. The computing device can connect to smart devices and control them based on user keywords, enabling home automation.

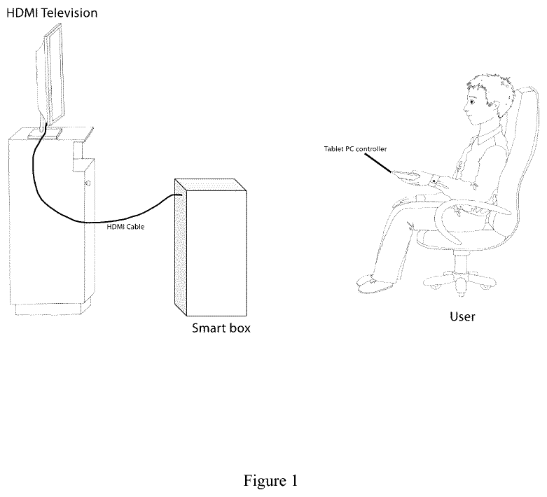

The proposed audio-visual conferencing system (shown in FIG. 1) enables humans/user especially the elderly/disable to engage and congenial with latest communication technology. The system includes a mobile computing device, a smart box having a data capturing unit and a display unit/output unit. Mobile computing device is a replica of a smartphone screen with buttons for letters/numbers and other features.

In an embodiment, mobile device is a tablet PC controller carried by the user for audio-visual conferencing. The mobile computing device is connected to smart box having data capturing unit using a wired or wireless communication network. In an embodiment, wireless network includes Bluetooth or infrared or Wi-Fi depending on the requirement of the user.

Smart box (shown in FIG. 1) is the heart of the system. It includes a stripped down to hold a data capturing unit such as mobile phone. The smart box also has an aperture to install camera and microphone of mobile phone. Smart phone camera look out from inside the smart box and capture the image of the elder in their living room/home and the microphone captures their voice.

In one of the embodiment the smart box (2) also has an additional inbuilt health port for measurement of Blood pressure (BP) machine and blood sugar of the person. The probes for sample collection can be attached to the device when necessary, through the health port, measurements taken and uploaded to the weekly/monthly health update report.

In one of the implementation a smartwatch is provided along with the system which is synched to the smart box (2), can be worn by the elder from time to time. In that time the vitals like pulse rate, oxygen saturation, Blood pressure etc. are logged in and transferred to the data acquisition unit wirelessly. The information is then compiled by the master app in the form of health update report which can be accessed online by the caregiver remotely.

In an embodiment, a repeated announcement or buzzer for medicine can be made for reminding elders to take medicine on time. The announcement stops only when the elder presses the confirmation button from mobile computing device.

Humidity/temperature indicator built within the device can be used to monitor and control the room temp. etc.

Hence, hardware & software-based video conferencing system enables humans especially the elderly to engage with latest technologies much more easily.

In an embodiment, the data capturing unit is a smart phone or a Rasberry PI chipset loaded with Android platform.

Further, the display unit receives voice and images of one or more users through HDMI cable and renders them in a conference call.

In an embodiment, the display unit is HDMI television to render the smart box output.

According to the present disclosure the physical components as used herein are described below:
Mobile computing device (1): A device equipped with an input module for receiving user inputs related to starting a video conferencing session and a first microprocessor to process the user inputs.
Data capturing unit (5): A unit coupled to the mobile computing device (1) and remotely placed. It consists of a camera (8), a microphone (9), and a second microprocessor (10) that controls the camera and microphone to capture the first user's voice and/or images and sends them to the server (3).
Server (3): Receives and stores the first user's voice and/or images from the data capturing unit (5) and also receives the second user's voice and/or images.
Communication network (51): The network that facilitates communication between the data capturing unit (5) and the server (3).
Output unit (4): An output device connected to the second microprocessor (10) that receives and renders the first user's and second user's voice and/or images.
Smart box (2): A box that houses the data capturing unit (5) and can be moved using a movement means (13).
Movement means (13): Such as a rotational unit (18), that allows the smart box (2) to move in three-dimensional space or rotate in a plane.
Positioning sensors (12): Sensors, that generate position data (19) of the first user and are used by the second microprocessor (10) to control the movement means (13) and move the smart box (2).
Security sensors (17): Sensors, such as heat sensors (31), smoke sensors (32), or motion sensors (33), that generate security data (34) to detect security issues.
Remote user device (29): A device used by the second user, which may include a controlling trigger (35) and a third microprocessor (55) for controlling the functionality of the system.
Data processing unit (37): A unit connected to the data capturing unit (5) that processes the first user's voice and/or images to enhance the quality.
Audio capturing device (38): An input unit in the mobile computing device (1) that captures audio as user input.
Speech detection unit (39): A unit in the mobile computing device (1) that processes audio to determine the first user's identification and/or identify keywords spoken by the first user.
Image capturing device (45): Device(s) connected to the first microprocessor (7) that captures images of the environment where the mobile computing device (1) is placed.
Motion sensor (33): Device(s) that capture motion data inside or outside the room where the mobile computing device (1) is placed.
Controller (50): A device that receives user controlling inputs (49) from the first microprocessor (7) and controls the smart devices (48) accordingly.
Microprocessors (7, 10, 55): Processors responsible for executing tasks and controlling various components of the system.
Memory unit (11): Stores historical and current health records of the first user (54, 56) for processing by the first microprocessor (7).
Voice recognition model (30): A model used by the second microprocessor (10) to recognize the voice of each first user based on captured audio (15).

According to the present disclosure the advanced communication system for senior care is designed to enhance the communication capabilities of elderly individuals who may require remote assistance or social interaction.

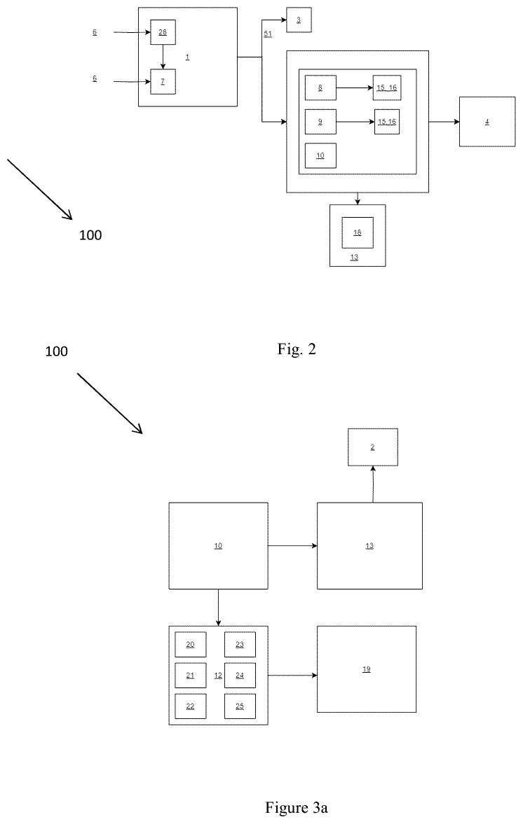

FIG. 2 illustrates system architecture diagram regarding video conferencing session as an embodiment of the present disclosure.

As illustrated in FIG. 2 this system (100) comprises a mobile computing device (1), a data capturing unit (5), a server (3), and an output unit (4) to facilitate seamless video conferencing sessions between seniors and their caregivers. Additionally, the system incorporates a smart box (2) equipped with a movement means (13) along with a rotational unit (18) to provide a versatile and interactive communication experience.

In one of the implementation the movement means can be electric motor, servo motor, gear mechanism etc.

In one of the embodiment the communication system (100) comprises a mobile computing device (1), such as a tablet or smartphone, which acts as the primary interface for the user (named as first user). It includes an input module (28) that allows the user to initiate a video conferencing session.

In one of the implementation the input module (28) could be keyboard, mouse, webcam, touchscreen, joystick etc.

The system (100) is equipped with a first microprocessor (7) responsible for processing user inputs (6) and transmitting them to other components of the system. The data capturing unit (5) is coupled to the mobile computing device (1) and remotely placed with respect to it. The data capturing unit (5) plays a crucial role in capturing audio (52) and visual data. Further, it includes a camera (8), a microphone (9), and a second microprocessor (10). The second microprocessor (10) receives user inputs (6) from the first microprocessor (7) and processes them to control the camera (8) and/or microphone (9), enabling the capturing of the first user's voice and/or images (15). The second microprocessor (10) is also connected to the server (3) via a communication network (51) to send the captured data and receive the second user's voice and/or images (16).

According to the present disclosure the server (3) acts as an intermediary between the data capturing unit (5) and the output unit (4).

The output unit (4), coupled with the second microprocessor (10) of the data capturing unit (5), is responsible for receiving and rendering audio and visual data. It enables users to see and hear the second user's voice and/or images (16) during the video conferencing session.

In one of the implementation the output unit can be a display screen, speakers, smart watch, smart television or a combination thereof, providing a comprehensive communication experience for the seniors.

In one of the embodiment, to enhance the mobility and interactivity of the system, the data capturing unit (5) can be placed inside a smart box (2). According to another embodiment, the smart box (2) can be coupled to a movement means (13), which allows it to be moved within the living space or other areas frequented by the senior. Furthermore, according to another embodiment of the present disclosure the movement means (13) may be a rotational unit (18), enables three-dimensional movement of the smart box (2). By utilizing the rotational unit (18), the data capturing unit (5) inside the smart box (2) can be rotated, allowing for adjustments in angle or position to capture a better view of the surroundings.

In another embodiment of the present disclosure the camera (8) of the system has auto focusing functionality.

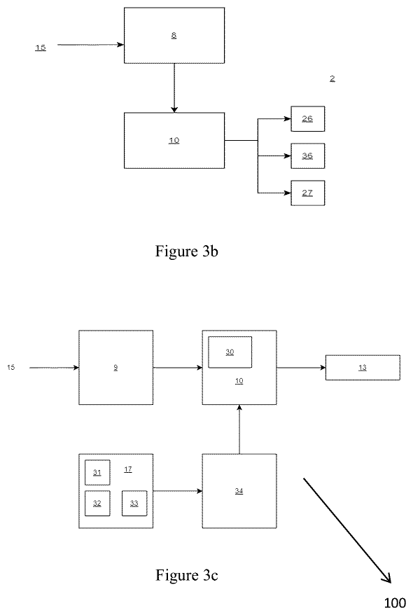

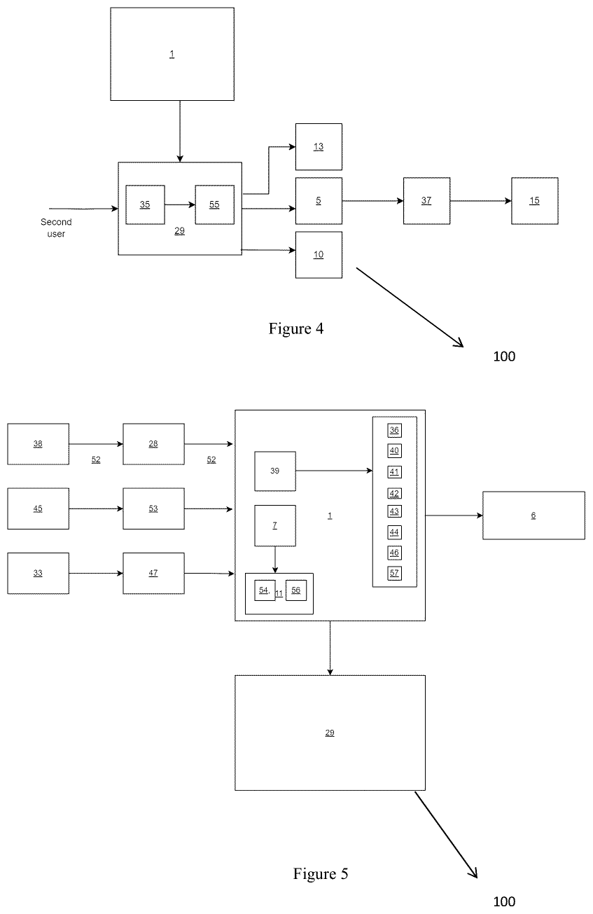

FIG. 3(a)-3(c) illustrate system architecture diagrams regarding autofocus of camera as an embodiment of the present disclosure.

In particular, FIG. 3a illustrates that the second microprocessor (10) is communicably coupled to one or more positioning sensors (12) that generate position data (19) of the first user. The second microprocessor (10) receives and processes the position data (19), allowing it to control the movement means (13) and adjust the position of the smart box (2) accordingly.

In one of the embodiments the positioning sensors (12) can be infrared sensors (20), image sensors (21), ultrasonic sensors (22), resistance-based sensors (23), capacitance-based sensors (24), optical sensors (25), or a combination thereof. These sensors contribute to accurately determining the position of the first user, ensuring precise movement control of the smart box (2).

FIG. 3(b) illustrates that, the camera captures images (15) of the first user, and the second microprocessor (10) processes these images (15). It determines whether the size of the body representation of the first user is smaller than a first threshold (26) and controls the camera (8) to zoom in. Similarly, if the size is greater than a threshold (27), the camera (8) zooms out. This intelligent camera control ensures optimal framing of the first user during video conferencing sessions.

In another embodiment the second microprocessor (10) identifies more than one first user and controls the camera (8) to zoom in or out individually for each user based on image processing. This feature allows for personalized video framing and improved visibility of each participant during the conference.

In another embodiment, the microphone (9) captures the voices (15) of each first user, and the second microprocessor (10) recognizes each user's voice using a voice recognition model (30). Based on voice recognition, the second microprocessor (10) controls the movement means (13) to focus the camera (8) on the first user whose voice is recognized at a particular instance. This ensures that the camera captures the corresponding user's face during conversations. FIG. 3c illustrates that the present disclosure further comprises security sensors (17) to capture relevant data within the system (100) itself. In another embodiment these sensors, such as heat sensors (31), smoke sensors (32), or motion sensors (33), generate security data (34). The second microprocessor (10) compares this data with predefined security criteria to detect and determine any security issues within the environment.

FIG. 4 illustrates system architecture diagram regarding remote controlling by a second user or remote user according to an embodiment of the present disclosure.

As illustrated in FIG. 4, as another embodiment of the present disclosure a remote user device (29) that is utilized by the second user, or by the mobile computing device (1), or a combination of both. This remote user device (29) is equipped with a controlling trigger (35) and a third microprocessor (55), which can establish a communicative connection with the data capturing unit (5), the movement means (13), the second microprocessor (10), or a combination thereof. The purpose of this arrangement is to enable the third microprocessor (55) to control one or more functionalities of the data capturing unit (5), the movement means (13), the second microprocessor (10), or a combination thereof.

In another embodiment this controlling trigger (35) can establish a communicative connection with the data processing unit (37) and is responsible for controlling the functionality of the data processing unit (37). In other words, the controlling trigger (35) allows the second user to manipulate the operations performed by the data processing unit (37) through the remote user device (29) or the mobile computing device (1).

Moreover, as illustrated in FIG. 4 the data capturing unit (5) is connected to a data processing unit (37) responsible for processing the voice and/or images (15) of the first user. The data processing unit (37) performs various operations such as de-noising the first user's voice (15), adjusting the contrast and/or brightness of the first user's image (15), or modifying the resolution of the user's images (15), or a combination thereof. These adjustments are made before transmitting the processed data to the second microprocessor (10).

FIG. 5 illustrates system architecture diagram regarding keywords identification and generating user input and passing information to a remote user as an embodiment of the present disclosure.

As illustrated in FIG. 5 the system (100) comprises an input unit (28), which in this case is an audio capturing device (38) designed to capture audio (52). The mobile computing device (1) is equipped with a speech detection unit (39) that receives and processes the captured audio (52). The purpose of the speech detection unit (39) is to determining the identification (36) of the first user and/or identifying the keywords (40) spoken by the first user within the audio (52). These keywords (40) are then processed to generate user input (6).

Moreover, the speech detection unit (39) is capable of identifying a keyword (40) as a distress keyword (41) or analyzing the captured sound to identify distress-related sound (57). If a distress keyword (41) or distress-related sound (57) is detected, the first microprocessor (7) records the captured audio (52) and sends it either to the remote user device (29) being used by the second user or to an emergency service provider device utilized by an emergency service provider. Further, the speech detection unit (39) is adapted to categorize the distress keyword (41) into pain-related keywords (42) or categorize distress-related sound (57) into pain-related sounds (43). When pain-related keywords (42) or pain-related sounds (43) are identified, the first microprocessor (7) receives and processes them to reach out to a healthcare service provider. Similarly, the speech detection unit (39) can categorize the distress keyword (41) into fear-related keywords (44) or categorize distress-related sound (57) into fear-related sounds (46). In this scenario, the first microprocessor (7) processes the fear-related keywords (44) or fear-related sounds (46) and initiates communication with a law enforcement service provider. These functionalities can be utilized individually or in combination.

According to the FIG. 4 the first microprocessor (7) is coupled with a memory unit (11) that stores the historical health record of the first user (54) and/or the current health record of the first user (56). The first microprocessor (7) can process the historical health record (54) and/or the current health record (56) along with the pain-related keywords (42) or pain-related sounds (43) to reach out to a healthcare service provider. According to another embodiment, the first microprocessor (7) is adapted to connect to one or more image capturing devices (45) that capture images (53) of the environment inside or outside the room where the mobile computing device (1) is located. Additionally, one or more motion sensor devices (33) capture motion data (47) inside or outside the room. The first microprocessor (7) processes the images (53) and/or the motion data (47) along with the fear-related keywords (44) or fear-related sounds (46) to detect intrusion and subsequently contacts a law enforcement service provider.

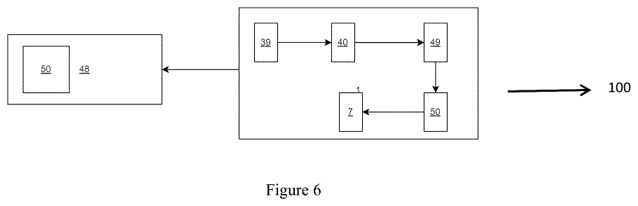

FIG. 6 illustrates system architecture diagram regarding control by other smart devices as one of the embodiment of the present disclosure.

As illustrated in FIG. 6 in one of the embodiment, the mobile computing device (1) is communicably connected to one or more smart devices (48). The speech detection unit (39) within the mobile computing device (1) is designed to identify keywords (40) that are relevant to controlling these smart devices (48). Once identified, the keywords (40) are processed to generate a user controlling input (49).

Further, the first microprocessor (7) receives the user controlling input (49) and communicates it to a controller (50) associated with the smart device (48).

In one of the implementation the smart device can be a mobile, laptop, television etc.

The controller (50) is specifically designed to process the user controlling input (49) received from the first microprocessor (7) and utilize it to control the respective smart device (48). Essentially, this arrangement allows the speech detection unit (39) to identify specific keywords (40) related to controlling the smart devices (48) through voice commands or spoken instructions. The first microprocessor (7) then facilitates the communication between the user's input (49) and the controller (50) of the smart device (48), enabling the user to control and interact with the smart device (48) using voice commands or spoken instructions recognized by the speech detection unit (39).

Advantage:

1. One on One Audio-Visual Communication Medium:

Currently making video calls using smartphones is very cumbersome with the small smartphone screen: difficult to view for a single person/multiple persons with the angle of smartphone placement needing to be continuously adjusted.

In Proposed system, the camera within the smart box captures most of the living room and multiple people easily. With the sound of distant person coming in over the speaker and the built in microphone picking up own voices for the distant person, conversation can be made without bending, crouching and looking at the small phone screen. It is like a meeting face to face. They can talk comfortably while sitting on the living room sofa or moving about. They can also see events like birthday celebrations etc. happening in the homes of caregivers live on their TV screens. This increases their engagement with their loved ones from a few minutes at a time to much longer.

2. One on One Engagement with their Assigned Care Manager.

Each elder has a care manager assigned who connects with them live for about an hour each day and chats with them about all kinds of things like current news/sports/politics/family affairs etc.

3. Group Chats with Other Elders.

Generally elders are confined to their homes and do not get a chance to talk to peers of a similar age group. The proposed system assign care manager to connect 6-7 elders on a group video chat where they call talk about the things that matter to them like current news/sports/politics/family affairs etc.

4. Peer—To—Peer Communication and Engagement with Multiple Friends/Family:

The video meeting/conference software accessed through the application can be used. All the persons in the conference will be seen on the large TV screen. This eliminates the need to view on a small phone or a laptop screen and the system enables people to connect, meet and chat comfortably.

5. Learning Application:

Using the device and the video meeting/conference software accessed through the application, a single instructor can take classes for multiple users concurrently. The classes could range from yoga, meditation, painting, music, chess, other courses and any other activity found useful. The tutors connect with groups of 6-8 elders at a time to teach these activities for about 1 hour per day.

The system user can participate using the built-in camera and speaker and perform activities normally without having to bend over a small phone screen. The instructor also can demonstrate live using his own system. Instructor sees the students/participants on the split screens on his own monitor and is able to teach/provide feedback as needed.

6. Mental Exercise Application:

These are very important for all humans especially for the elderly. Using the application within the system, multiple mental exercises and games can be accessed and undertaken. These could range from chess, crosswords, puzzles, IQ/current affairs and other quizzes, and many more activities that can be embedded depending the need of the users. The user uses the remote to control the activity and can engage comfortably without the need to look into a small screen.

7. General Entertainment Application:

Users can access multiple entertainment avenues using the applications withing the system including playlists of songs, live concert feeds. Live interactive games like Tambola/song games can also be engaged by multiple users on their own as well as a live host who will conduct for the group. The elders can use the master app to launch different apps for entertainment like music/news/current affairs etc.

8. Reading:

The master app launches several apps which contain thousands of books and the elders can read them comfortably on their large TV screen.

9. Security/Monitoring/Misc Applications:

The device camera can be left on with the live feed being constantly monitored by the distant family member or friend. The feed can also be monitored if the patient is sick and bedridden and may need assistance anytime. The distant family member/friend is thus assured of the well-being and safety of the user. This is only possible through the device mounted camera and is virtually impossible to do using a smartphone. Other security and safety features like smoke detector, infrared beam for intrusion detection can be installed within the device depending on the requirement of the users. The caregiver can monitor the elder and their home through the built in camera in the smart box. This is especially useful when the elder is unwell where the caregiver can see the doctor examination etc.

### LIST OF REFERENCE NUMERALS

1—system
2—Smart box
3—Server
4—Output unit
5—data capturing unit
6—user input
7—First Microprocessor
8—Camera
9—Microphone
10—Second Microprocessor
11—Memory unit
12—Sensor panel
13—Movement means/servomotor
14—swivel base
15—first user's voice and/or images
16—Second user's voice and/or images
17—security sensors
18—rotational unit
19—position data
20—infrared sensors
21—image sensors
22—ultrasonic sensors
23—resistance-based sensors
24—capacitance-based sensors
25—optical sensors
26—first threshold (related to the size of body representation)
27—threshold (related to the size of body representation)
28—input module/unit
29—Remote user device
30—recognition model
31—Heat sensor
32—Smoke sensor
33—Motion sensor
34—Predefined security data
35—Controlling trigger
36—Determine identification of first user
37—Data processing unit
38—Audio capturing device
39—Speech detection unit
40—Keywords
41—distress keyword
42—pain related keyword
43—pain related sound
44—fear related keyword
45—Image capturing device
46—fear related sound
47—Motion data
48—Smart device
49—User controlling input
50—Controller
51—Communication network
52—audio
53—capturing images
54—historical health record of the first user
55—Third microprocessor
56—current health record of the first user
57—distress related sound

1. An advanced communication system (100) for senior care comprising:
a mobile computing device (1) having an input module (28) for receiving a user input (6) related to starting a video conferencing session, and a first microprocessor (7) adapted to receive the user input (6);
a data capturing unit (5) coupled to the mobile computing device (1) and remotely placed with respect to the mobile computing device (1), and a server (3) through a communication network (51), the data capturing unit (5) includes a camera (8), a microphone (9), and a second microprocessor (10), wherein the second microprocessor (10) is adapted to receive the user input (6) from the first microprocessor (7), the second microprocessor (10) is adapted to process the user input (6) to control the camera (8) and/or microphone (9) to start or stop capturing one or more first user's voice and/or images (15), the second microprocessor (10) is adapted to be connected to the server (3) to send one or more first user's voice and/or images (15), and to receive one or more second user's voice and/or images (16);
an output unit (4) coupled with the second microprocessor (10) and is adapted to receive and render:
one or more first user's voice and/or images (15),
one or more second user's voice and/or images (16),
or combination thereof.

2. The system (100) as claimed in claim 1, wherein the data capturing unit (5) is placed inside a smart box (2), and the smart box (2) is coupled to a movement means (13) to move the smart box (2).

3. The system (100) as claimed in claim 2, wherein the movement means (13) is a rotational unit (18) adapted to rotate the smart box (2) in three-dimensional space.

4. The system (100) as claimed in claim 3, wherein the rotational unit (18) is connected to one of the surface of the smart box (2), and adapted to rotate the smart box (2) in a plane.

5. The system (100) as claimed in claim 2, wherein the movement means (13) is connected to a second microprocessor (10) which controls the movement means (13) to move the smart box (2), and the second microprocessor (10) is communicably coupled to one or more positioning sensors (12), the position sensors (12) are adapted to generate a position data (19) of the one or more first user, and the second microprocessor (10) is adapted to receive and process the position data (19) and adapted to control the movement means (13) to move the smart box (2).

6. The system (100) as claimed in claim 5, wherein one or more position sensors (12) are infrared sensors (20), image sensors (21), ultrasonic sensors (22), resistance-based sensors (23), capacitance-based sensors (24), or optical sensors (25), or combination thereof.

7. The system as claimed in claim 5, wherein the camera (8) is adapted to capture one or more images (15) of the one or more first user, the second microprocessor (10) is adapted to process the images (15) and is adapted to determine if a size of body representation of the one or more first user are lesser than a first threshold (26) and to control the camera (8) to zoom in the lens of the camera (8), and to determine if the size of body representation of the one or more first user are greater than a threshold (27) and to control the camera (8) to zoom out the lens of the camera (8).

8. The system (100) as claimed in claim 7, wherein if the second microprocessor (10) is adapted to determine identification (36) of more than one first users in the environment where the smart box (2) is placed and who are located at different locations in the room, the second microprocessor (10) is adapted to control the camera (8) to zoom in or zoom out individually for each of the first users based on processing of the images (15) captured by the camera (8) for each of the first user.

9. The system as claimed in claim 8, wherein the microphone (9) is communicably coupled to the second microprocessor (10), and adapted to capture voices (15) of each of the first users, the second microprocessor (10) is adapted to recognize voice (15) of each first user based on a voice recognition model (30), and adapted to control the movement means (13) to move the smart box (2) to focus the camera (8) onto the first user whose voice is recognized at a particular instance.

10. The system (100) as claimed in claim 5 comprising one or more security sensors (17) adapted to generate a security data (34), the security sensors (17) includes a heat sensor (31), a smoke sensor (32), or a motion sensor (33), or combination thereof, the security sensors (17) are coupled to the second microprocessor (10), and the second microprocessor (10) is adapted to compares the security data (34) with a predefined security data (34) to determine a security issue.

11. The system as claimed in claim 2, wherein a remote user device (29) being used by the second user, or the mobile computing device (1), or combination thereof comprises a controlling trigger (35), a third microprocessor (55) adapted to be communicably coupled to the data capturing unit (5), the movement means (13), the second microprocessor (10), or combination thereof for controlling one or more functionality of the data capturing unit (5), the movement means (13), the second microprocessor (10), or combination thereof.

12. The system as claims in claim 11, wherein the data capturing unit (5) is coupled to a data processing unit (37) adapted to process the first user's voice and/or images (15) to de-noise the first user's voice (15), to tune contrast and/or brightness of the first user's image (15), or to tune resolution of the user's images (15), or combination thereof before sending it to the second microprocessor (10).

13. The system as claimed in claim 11, wherein the remote user device (29) being used by the second user, or the mobile computing device (1), or combination thereof comprises a controlling trigger (35) adapted to be communicably coupled to the data processing unit (37) for controlling functionality of the data processing unit (37).

14. The system (100) as claimed in claim 1, wherein the input unit (28) is an audio capturing device (38) adapted to capture audio (52), and the mobile computing device (1) comprises a speech detection unit (39) adapted to receive and process the audio (52) to determine identification (36) of the first user and/or to identify keywords (40) spoken by the first user in the audio (52), and to process the keywords (40) to generate the user input (6).

15. The system (100) as claimed in claim 14, wherein the speech detection unit (39) is adapted to identify the keyword (40) as a distress keyword (41) or to process the captured sound to identify distress related sound (57), the first microprocessor (7) is adapted to record the captured audio (52) and to send to the remote user device (29) being used by the second user or to an emergency service provider device being used by an emergency service provider.

16. The system (100) as claimed in claim 15, wherein the speech detection unit (39) is adapted:
to further categorize the distress keyword (41) to pain related keyword (42) or to categorize distress related sound (57) to pain related sound (43), the first microprocessor (7) is adapted to receive and process the pain related keyword (42) or the pain related sound (43) and to reach out to a healthcare service provider, or
to further categorize the distress keyword (41) to fear related keyword (44) or to categorize distress related sound (57) to fear related sound (46), the first microprocessor (7) is adapted to receive and process the fear related keyword (44) or the fear related sound (46) and to reach out to a law enforcement service provider, or combination thereof.

17. The system (100) as claimed in claim 16, wherein the first microprocessor (7) is coupled to a memory unit (11) storing a historical health record of the first user (54) and/or a current health record of the first user 56), to process the historical health record of the first user (54) and/or the current health record (56) along with the pain related keyword (42) or the pain related sound (29) and to reach out to a healthcare service provider.

18. The system (100) as claimed in claim 16, the first microprocessor (7) is adapted to connect to one or more image capturing device (45) capturing images (53) of environment inside or outside a room where the mobile computing device (1) is placed and/or one or more motion sensor (33) devices capturing motion data (47) inside or outside a room where the mobile computing device (1) is placed, to process the images (53) and/or the motion data (47) along with the fear related keyword (44) or the fear related sound (46) to detect intrusion and further to reach out to a law enforcement service provider.

19. The system (100) as claimed in claim 14, wherein the mobile computing device (1) is communicably coupled to one or more smart devices (48), and the speech detection unit (39) is adapted to identify keywords (40) related to controlling the smart devices (48), and to process the keywords (40) to generate a user controlling input (49), and the first microprocessor (7) is adapted to receive the user controlling input (49) and communicate the same to a controller (50) of the device, and the controller (50) is adapted to process the user controlling input (49) to control the smart device (48).
